(19) United States
(12) Patent Application Publication
(10) Pub. No.: US 2015/0099981 A1
(43) Pub. Date: Apr. 9, 2015

(54) Tubular Light Guide

(71) Applicant: United Sciences, LLC, Atlanta, GA (US)

(72) Inventors: SCOTT CAHALL, Fairport, NY (US); EOHAN GEORGE, Atlanta, GA (US); KAROL HATZILIAS, Atlanta, GA (US); ROD MCAFEE, Atlanta, GA (US); WESS ERIC SHARPE, Atlanta, GA (US)

(21) Appl. No.: 14/049,542

(22) Filed: Oct. 9, 2013

(51) Int. Cl. A61B 5/107; A61B 5/00

(52) U.S. Cl. CPC A61B 5/1079; A61B 5/742
USPC 600/476

(57) ABSTRACT

Disclosed are various embodiments for scanning a cavity surface using a tubular element comprising an inner wall and an outer wall. The tubular element may be designed to guide and approximately collimate light received from a light source from the first end of the tubular element to the second end of the tubular element. The light may be guided between the inner wall and the outer wall of the tubular element. The light may be projected from the tubular element onto a cone mirror fixed at the second end of the tubular element. The cone mirror may be designed to radially reflect the light guided to the second end of the tubular element when the light is within a predefined wavelength range.

### CROSS REFERENCE TO RELATED APPLICATIONS

This application is related to U.S. patent application Ser. No. ______, filed on Oct. ______, 2013 (Attorney Docket No. 52105-1020) and entitled “Tapered Optical Guide,” U.S. patent application Ser. No. ______, filed on Oct. ______, 2013 (Attorney Docket No. 52105-1030) and entitled “Display for Three-Dimensional Imaging,” U.S. patent application Ser. No. ______, filed on Oct. ______, 2013 (Attorney Docket No. 52105-1040) and entitled “Fan Light Element,” U.S. patent application Ser. No. ______, filed on Oct. ______, 2013 (Attorney Docket No. 52105-1050) and entitled “Integrated Tracking with World Modeling,” U.S. patent application Ser. No. ______, filed on Oct. ______, 2013 (Attorney Docket No. 52105-1060) and entitled “Integrated Tracking with Fiducial-based Modeling,” U.S. patent application Ser. No. ______, filed on Oct. ______, 2013 (Attorney Docket No. 52105-1070) and entitled “Integrated Calibration Cradle,” and U.S. patent application Ser. No. ______, filed on Oct. ______, 2013 (Attorney Docket No. 52105-1080) and entitled “Calibration of 3D Scanning Device,” all of which are hereby incorporated by reference in their entirety.

### BACKGROUND

There are various needs for understanding the shape and size of cavity surfaces, such as, for example, body cavities. For example, hearing aids, hearing protection, and custom head phones often require silicone impressions to be made of a patient's ear canal. Audiologists inject the silicone material into an ear, wait for it to harden, and then provide the mold to manufacturers who use the resulting silicone impression to create a custom fitting in-ear device. The process is slow, expensive, inconsistent, and unpleasant for the patient, and can even be dangerous as injecting silicone risks affecting the ear drum.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the disclosure. Moreover, in the drawings, like reference numerals designate corresponding parts throughout the several views.

FIGS. 1A-1B are graphical representations of examples of a scanning device in accordance to various embodiments of the present disclosure.

FIGS. 2A-2D are graphical representations of examples of a scanning probe mounted to the scanning device of FIGS. 1A-1B in accordance with various embodiments of the present disclosure.

FIG. 3 is a graphical representation of a 360 degree ring of light projected from the scanning probe of FIGS. 2A-2D in accordance to various embodiments of the present disclosure.

FIG. 4 is a graphical representation of a fan light element mounted to the scanning device of FIG. 1A in accordance to various embodiments of the present disclosure.

FIG. 5 is a graphical representation of a single element lens emitting light generated by a light source of FIG. 4 in accordance to various embodiments of the present disclosure.

FIG. 6 is a graphical representation of a fan line of light projected from a fan light element of FIG. 4 in accordance to various embodiments of the present disclosure.

FIGS. 7A and 7B are graphical representations of an optical guide of the scanning probe as shown in FIGS. 2A-2D in accordance with various embodiments of the present disclosure

FIG. 8 is a flowchart illustrating one example of scanning and constructing scanned images by the scanning device of FIGS. 1A-1B in accordance with various embodiments of the present disclosure.

FIG. 9 is a flowchart illustrating one example projecting video illuminating light by the scanning device of FIGS. 1A-1B in accordance with various embodiments of the present disclosure.

### DETAILED DESCRIPTION

The present disclosure relates to a tubular light guide. Through the use of optical technology, a portable scanning device may be designed to determine the shape of surfaces, including, but not limited to, cavity surfaces. Further, the portable scanning device may be designed to determine the shape of cavity surfaces, including cavity surfaces defining body cavities, such as the size of a shape of an ear canal, throat, mouth, nostrils, or intestines of a body. For example, the scanning device may be able to construct a three-dimensional (3D) image and shape of an ear canal through the use of a tubular light guide. Tubular light guides may direct light from a light source onto a cavity surface for imaging. In the following discussion, a general description of the system and its components is provided, followed by a discussion of the operation of the same.

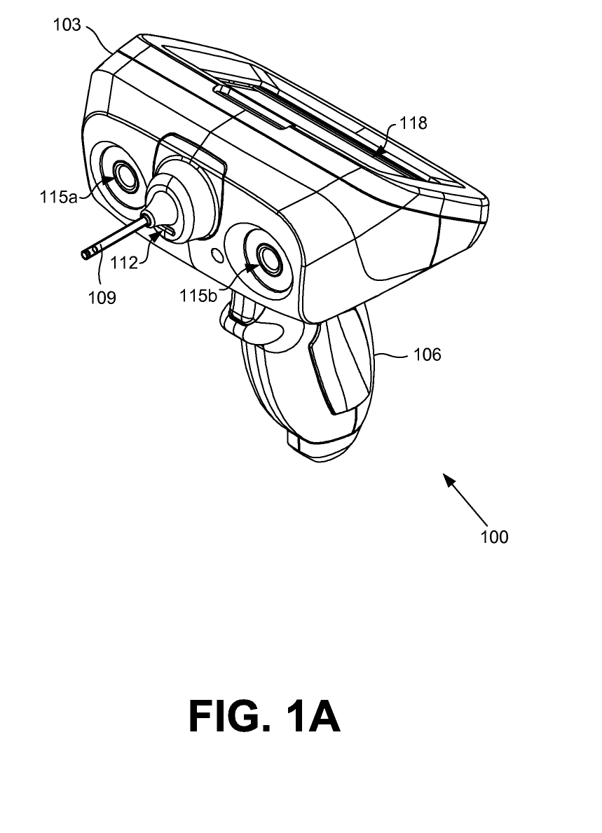

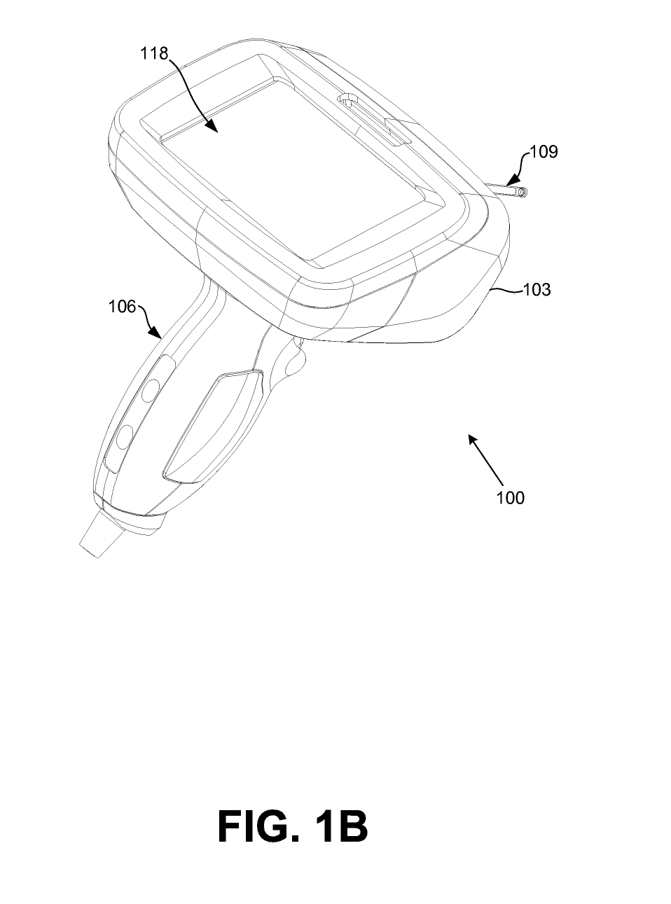

With reference to FIG. 1A, shown is a drawing of an example of a scanning device 100 according to various embodiments. The scanning device 100 as illustrated in FIG. 1A includes a body 103 and a hand grip 106. Mounted upon the body 103 of the scanning device 100 are a probe 109, a fan light element 112, tracking sensors 115a, 115b and a display screen 118. The body 103 may also have mounted within it an image sensor for reconstructing captured images and reflections when the scanning device is used to scan a surface. The hand grip 106 may be configured such that the length is long enough to accommodate large hands and the diameter is small enough to provide enough comfort for smaller hands.

As will be discussed in further detail below, the probe 109 is configured to guide light received at a proximal end of the probe 109 to a distal end of the probe 109. The light may be radially reflected forming a 360 degree ring and/or projected from the tip of the probe 109. In some embodiments, light guided to the distal end of the probe 109 is radially reflected forming an unbroken 360 degree ring. The scanning device 100 may be configured to scan a cavity surface by projecting the 360 degree ring onto the cavity surface and capturing reflections from the projected ring to reconstruct the image and shape of the cavity surface. In addition, the scanning device 100 may be configured to capture video images of the cavity surface by projecting video illuminating light onto the cavity surface and capturing video images of the cavity surface.

The fan light element 112 mounted onto the scanning device 100 may be configured to emit light in a fan line for scanning an outer surface. The fan light element 112 comprises a fan light source projecting light onto a single element lens to collimate the light and generate a fan line for scanning the outer surface. By using triangulation of the reflections captured when projected onto a surface, the imaging sensor within the scanning device 100 may reconstruct the scanned surface.

FIG. 1A illustrates an example of the tracking sensors 115a, 115b mounted on the body 103 of the scanning device 100 in an orientation that is opposite from the display screen 118. In some embodiments the tracking sensors 115a, 115b are oriented so that they can sense reflections of tracking illumination from tracking targets fixed in a position with respect to a scanned surface. In some embodiments, the tracking targets may be artificial targets that may be positioned in an area relative to the cavity to be scanned. For example, if a person's ear is being scanned, then the tracking targets may be positioned on the person's head. In other embodiments, the tracking targets may be naturally occurring features surrounding and/or within the cavity to be scanned. For example, still assuming that a person's ear is being scanned, the tracking targets may include, hair, folds of the ear, skin tone changes, freckles, moles, and/or any other naturally occurring feature on the person's head relative to the ear.

The tracking illumination may be infrared, white light, and/or any other consistent type of illumination. However, it should be noted that the use of infrared reduces the amount of light observed by the operator of the scanning device 100 and/or the person being scanned. The tracking sensors 115a, 115b may be configured to detect the reflections of the illuminated light, e.g., infrared, from the tracking targets.

The display screen 118 may include video images of the cavity captured by the image sensor within the scanning device 100 as the probe 109 is moved within the cavity. The display screen 118 may also display, either separately or simultaneously, real-time reconstructions of 3D images corresponding to the scanned cavity.

Shown in FIG. 1B is another view of the scanning device 100 according to various embodiments. The scanning device 100 includes the body 103, the probe 109, the display screen 118, and the hand grip 106, all implemented in a fashion similar to that of the scanning device described above with reference to FIG. 1A. The display screen 118 is positioned on the body 103 in relation to the probe 109 so that when the probe 109 is positioned for scanning, both the display screen 118 and the probe 109 are visible to an operator of the scanning device 100. In the examples of FIGS. 1A and 1B, the scanning device 100 is implemented with the probe 109 mounted on the body 103 between the hand grip 106 and the display screen 118. The display screen 118 is mounted on the opposite side of the body 103 from the probe 109 and distally from the hand grip 106. In this way, when an operator takes the hand grip 106 in the operator's hand and positions the probe to scan a surface, both the probe 109 and the display screen 118 are easily visible at all times to the operator.

The display screen 118 is coupled for data communications to an image sensor, and the display screen 118 displays images of the scanned surface. The displayed images may include video images of the cavity captured by the image sensor as the probe is moved within the cavity. The displayed images may also include real-time constructions of 3D images corresponding to the scanned cavity. The display screen 118 may be configured to, either separately or simultaneously, display the video images and the 3D images.

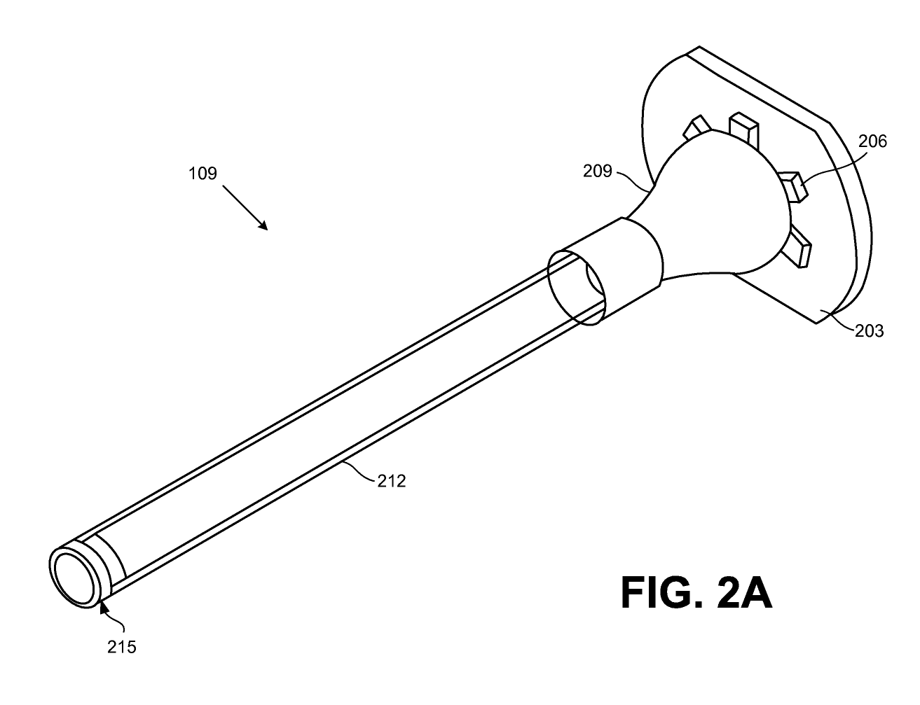

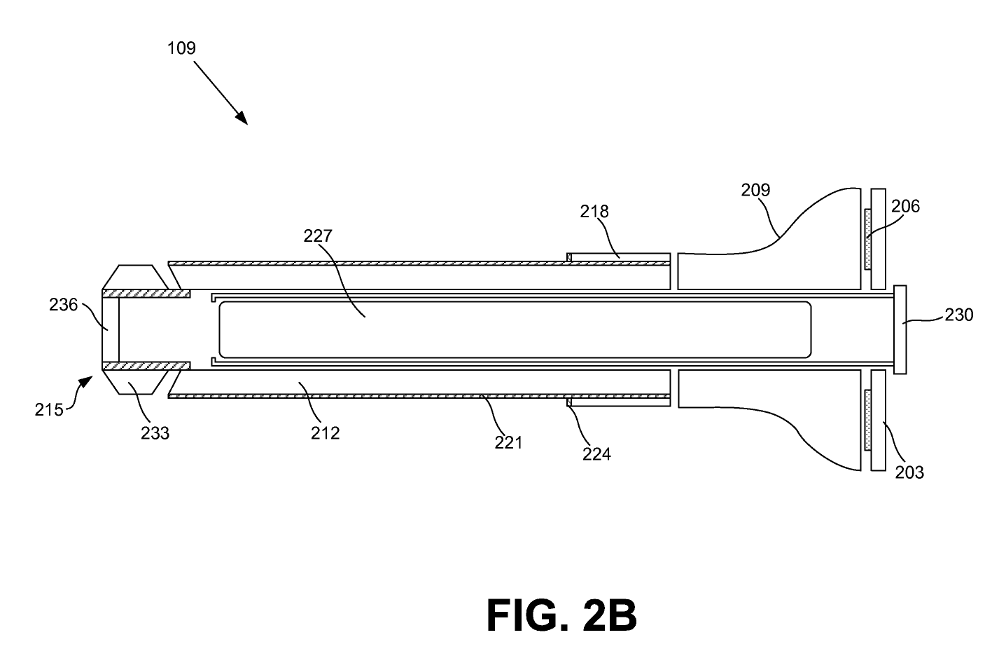

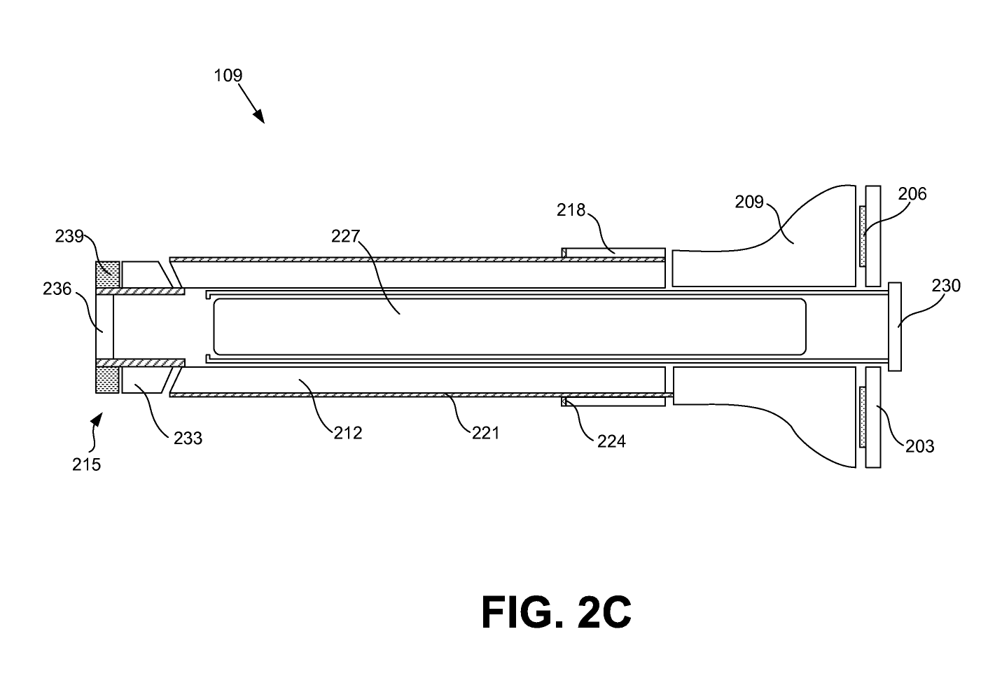

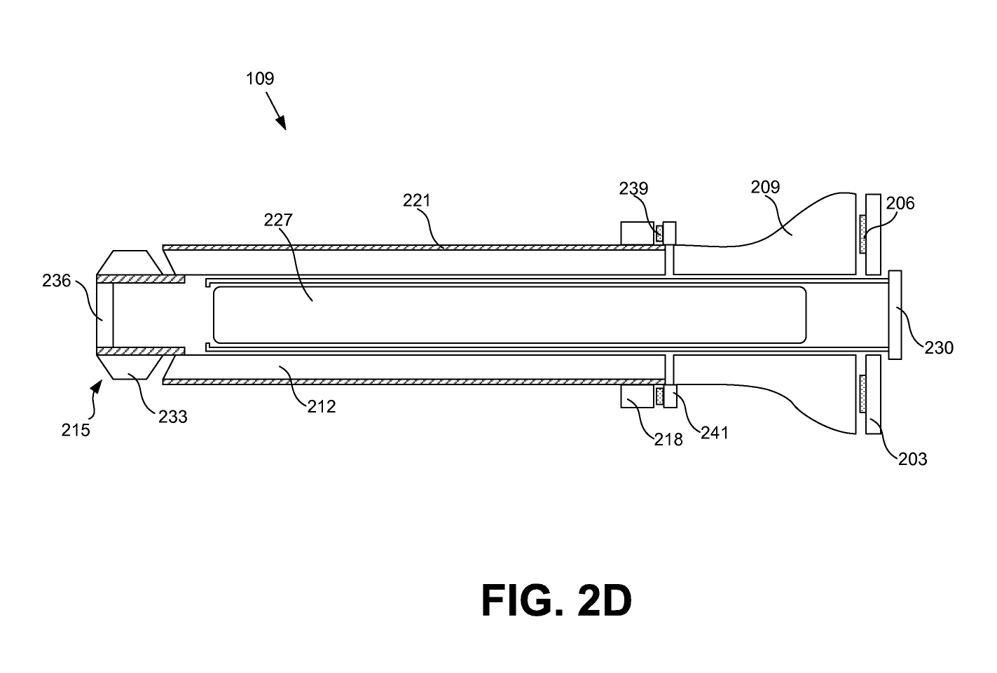

Turning now to FIG. 2A, shown is an example of the probe 109 according to various embodiments. In some embodiments, the probe 109 may include a lighting element 203, a light source 206, an optical guide 209, a tubular element 212, a probe tip 215, and/or other elements not illustrated. The probe 109 is designed to guide and approximately collimate light generated by the light source 206 through the tubular element 212 for projection onto a cavity surface. The light may be used for video illumination and/or scanning of the cavity surface.

The lighting element 203 may include one or more light sources 206, as illustrated in FIG. 2A. The light source 206 may comprise a light emitting diode (LED), laser, and/or other appropriate type of light source. In instances where the lighting element 203 includes multiple light sources 206, the light sources 206 may be of the same type such that each is configured to generate light within similar wavelengths. However, in some embodiments the lighting element 203 may comprise a light source 206 that may generate light within a first wavelength range (e.g. about 450 nm and less) and another light source 206 that may generate light within a second wavelength range (e.g. about 500 nm and above). For example, the lighting element 203 may comprise a blue LED configured to generate a blue light and a white LED configured to generate a white light. Accordingly, one light source 206 (e.g., the blue LED) may generate light for scanning a surface cavity while the other light source 206 (e.g., the white LED) may generate light used for video illumination of the surface cavity. Additionally, in some embodiments, the light sources 206 may be configured to alternately generate light such that only one type of light source 206 is generating light at any given instance.

The lighting element 203 is configured to position the light source 206 on the first end of the optical guide 209. While the lighting element 203, as shown in FIG. 2A, comprises a substantially circular shape, the lighting element 203 may be designed in any shape so long as light generated by the light source 206 may be received at the first end of the optical guide 209. In some embodiments, the lighting element 203 may comprise a printed circuit board (PCB) and/or other type of supporting element that may include the one or more light sources 206. In other embodiments, the light source 206 may be coupled directly to the optical guide 209 without the use of the lighting element 203.

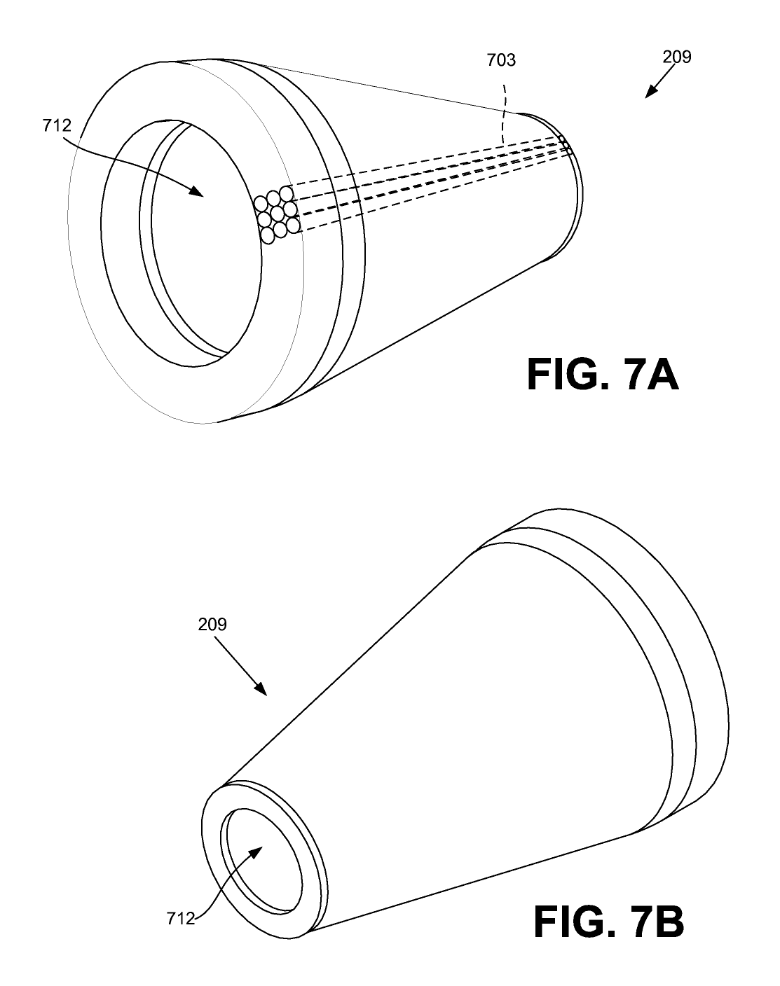

The optical guide 209 is configured to guide light generated by the light source 206 to the proximal end of the tubular element 212. As will be discussed in greater detail with respect to FIGS. 7A and 7B, the optical guide 209 may comprise a plurality of optical fibers. Each of the optical fibers may be designed to guide light received from the light source 206 at a first terminal end of each of the optical fibers to a second terminal end of the optical fibers. The optical guide 209 may be tapered such that a diameter of the first end is greater than a diameter of the second end of the optical guide 209. As such, the tapering for the optical guide 209 compensates for a size difference between the light source 206 and the tubular element 212. For example, assume that the light source 206 is approximately 1600 microns wide and the tubular element is approximately 250 microns wide. The optical guide 209 may be tapered so that the diameter of the first end of the optical guide is approximately 1600 microns (e.g. width of light source) and the diameter of the second end of the optical guide is approximately 250 microns wide (e.g. width of tubular element 212). Additionally, coupling loss may be reduced when projecting light into the tubular element 212.

The second end of the optical guide 209 is positioned adjacent to the proximal end of the tubular element 212. The optical guide 209 may be coupled to the tubular element 212 via index matching and optically transparent glue relative to the tubular element 212, epoxy, and/or other type of affixing material that is optically transparent. In some embodiments, the optical guide 209 and tubular element 212 may not be completely or even partially bonded allowing for air to separate the optical guide 209 from the tubular element 212. However, it should be noted that for minimizing the amount of escaping light, an index matching optically transparent epoxy or glue may be the most efficient material for bonding the optical guide 209.

As previously discussed, the second end of the optical guide is disposed adjacent to the proximal end of the tubular element 212. A probe tip 215 is disposed adjacent to the distal end of the tubular element. As will be discussed in greater detail with respect to FIG. 2B, the light exiting the tubular element 212 may be radially reflected at the probe tip 215 for scanning or may be passed though the probe tip 215 for video illumination. The tubular element 212 may be constructed of glass, acrylic, or any other type of material that may be used to guide light. The tubular element 212 is a tube having an inner wall and an outer wall. In addition, the tubular element 212 comprises a channel defined by the inner wall of the tube and extending from the proximal end to the distal end of the tubular element 212. The tubular element 212 may be designed to guide light received from the optical guide 209 between the inner wall and the outer wall of the tubular element 212 to the distal end of the tubular element 212.

In some embodiments, the outer wall of the tubular element 212 may comprise a cladding. The cladding comprises a refractive index material that is lower than the index of the material of the tubular element 212. In other embodiments, both the inner wall and the outer wall comprise a cladding. Accordingly, the cladding on the inner wall and the outer wall of the tubular element 212 forms a clad-core-clad configuration. The light is guided through the tubular element 212 within the core and the cladding may be added to help the amount of light that may escape from the tubular element 212. Additionally, the cladding configuration approximately collimates the light being guided to the second end of the tubular element 212. The amount of light that is internally reflected within the inner walls and outer walls of the tubular element 212 may be based at least upon the numerical aperture of the tubular element 212. For example, the numerical aperture may be controlled based on a ratio of the index of refraction between the cladding material and the material of the tubular element 212. The amount of light that escapes the core defined by the inner wall and/or outer wall of the tubular element 212 is dependent upon the numerical aperture of the tubular element 212. Additionally, the exit angle at which the light exits the tubular element 212 may be reduced. As such, the light transmitted through the tubular element 212 becomes approximately collimated as it is guided to the second end of the tubular element 212. Accordingly, the light projected from the tubular element 212 is an approximately collimated beam of light. The numerical aperture may be in the range of 0.4 or less, 0.2 or less, or 1.4 or less. Further, to improve the transfer of light from the optical guide 209 to the first end of the tubular element 212, the numerical aperture of the optical fibers 703 (FIG. 7A) of the optical guide 209 may approximately match the numerical aperture of the tubular element 212.

In some embodiments, the tubular element 212 may be designed such that one or more frustration masks 221 (FIG. 2B) surround at least a portion of the outside of the tubular element 212. The frustration mask 221 may comprise, but is not limited to, opaque glue, one or more blackening agents (e.g. Acktar), electrical tape (with index matching adhesive), and/or any other type of material that absorbs light. While some of the embodiments may not include any type of frustration mask 221, it should be noted that the more frustration that is included along the length of the tubular element 212, the more collimated the light will be at the second end of the tubular element 212. For example, since the frustration absorbs the light escaping the tubular element, the light that is transmitted to the second end of the tubular element will be approximately collimated.

The length of the tubular element 212 may be defined by the length needed to scan a cavity surface. For example, if the cavity surface is an ear canal, the length of the tube may be defined based on the least intrusive length needed to accurately and safely scan the ear canal. For example, the length of the probe 109 may be about 30 mm, and the width between the inner wall and the outer wall may be about 250 microns. While the width of the outer wall and inner wall of the tubular element 212 is not limited, it should optimally be designed as thin as practicable.

Referring next to FIG. 2B, shown is a more detailed illustration of the probe 109 according to various embodiments. The probe 109, as shown in FIG. 2B, includes the lighting element 203, the light source 206, the optical guide 209, the tubular element 212, an illumination tube 218, a frustration mask 221, a filter element 224, a lens system 227, an image sensor 230, and/or other elements. As illustrated in FIG. 2B, the probe tip 215 may comprise a cone mirror 233, distal cone mask 234, and a distal window 236.

As shown in FIG. 2B, the probe 109 comprises the illumination tube 218. The illumination tube 218 may comprise a tube having an inner wall and an outer wall. The illumination tube 218 may be comprised of glass, acrylic, and/or other material that can be used to guide light. In various embodiments, the illumination tube 218 may be disposed around the proximal end of the tubular element 212. In some embodiments, the illumination tube 218 may be disposed around the tubular element 212 over a frustration mask 221, such as, for example, an index matching opaque glue. In other embodiments, the illumination tube 218 may be directly disposed around to the tubular element 212. Further, in other embodiments, the probe 109 may be designed without an illumination tube 218.

The illumination tube 218 may guide light to be projected from the probe 109 for video illumination. In some embodiments, the illumination tube 218 may include a filter element 224. The filter element 224 may include a material, such as, for example, a dichroic material used to reflect light within a predefined wavelength range such that only the video illumination light may be projected from the probe 109 via that illumination tube 218. For example, if multiple light sources 206 are coupled to the lighting element 203, and one light source 206 generates light for video illumination and another light source 206 generates light for scanning, the filter element 224 may be designed to pass only light generated by the one light source 206 that generates the video illuminating light.

The illumination tube 218 may be configured to receive light generated by the light source 206 coupled to the lighting element 203. Accordingly, the diameter of an end of the optical guide 209 that is disposed adjacent to the illumination tube 218 and tubular element 212 may be greater than the diameter of the tubular element 212. In such embodiments, the diameter of the end of the optical guide 209 that is disposed adjacent to the illumination tube 218 and tubular element 212 may be substantially equal to the diameter of the illumination tube 218 disposed around the tubular element 212. As such, light guided through the optical fibers of the optical guide 209 may project into the illumination tube 218. In embodiments where the illumination tube 218 comprises a filter element 224, the filter element 224 may reflect light back into the optical guide 209 if the light is within a predefined wavelength range. Otherwise, the filter element 224 may allow the light to pass through for projection from the illumination tube 218. Although the filter element 224 is shown at the distal end of the illumination tube 218 in FIG. 2B, it should be noted that in some embodiments, the filter element 224 may be located at other portions along the illumination tube 218. Additionally, the illumination tube 218 may be designed without a filter element 224 thereby projecting any light received.

The tubular element 212 as illustrated in FIG. 2B includes a frustration mask 221 surrounding the outer wall of the tubular element 212. While the frustration mask 221 in FIG. 2B is shown to surround the entire outer surface of the tubular element 212, it should be noted that the frustration mask 221 may only cover a portion of the tubular element 212. In some embodiments, the tubular element 212 may not have a frustration mask 221 disposed on the tubular element 212. In addition, in some embodiments, the frustration mask 221 disposed between the illumination tube 218 and the tubular wall may comprise one type of frustration (e.g. opaque glue), while the remainder of the tubular element 212 may comprise another type of frustration (e.g. a blackening agent). As previously discussed, the more frustration surrounding the outer wall of the tubular element 212, the more collimated the light will be at the second end of the tubular element 212.

The tubular element 212 may be tapered at the distal end for coupling to the cone mirror 233. The tubular element 212 may be bonded to the cone mirror 233 via index matching and optically transparent glue, epoxy, and/or other type of optically transparent affixing material. In some embodiments, the tubular element 212 and the cone mirror 233 are not bonded. Accordingly, air may separate the tubular element 212 from the cone mirror 233. However, this embodiment is not preferred as light may escape. In addition, as illustrated in FIG. 2B, the cone mirror 233 may comprise a channel extending from a proximal end of the cone mirror 233 to a distal end of the cone mirror 233.

The portion of the cone mirror 233 surrounding the channel may comprise a distal cone mask 234 extending from the inner wall of the cone mirror 233 to at least a portion of the distal end of the inner wall of the tubular element 212. The distal cone mask 234 is configured to absorb the light projected from the tubular element 212 and minimize the amount of light that may escape into the distal window 236 and onto the lens system 227. For example, without the use of the distal cone mask 234 some of the light guided through the tubular element 212 may leak into the distal window 236 generating a ring projected from the distal window 236. Accordingly, the reconstructed image would include the ring based on the captured reflections of the projected ring. Additionally, without the use of the distal cone mask 234, some of the light guided through the tubular element 212 may escape and be in direct view of the lens system 227. Accordingly, the lens would be capturing the direct light and not the reflections of the light when projected onto a surface.

Light guided through the tubular element 212 may be projected onto the cone mirror 233. The cone mirror 233 may be configured to radially reflect the light received from the tubular element 212 forming an unbroken 360 degree ring of light. In some embodiments, the cone mirror 233 may comprise a type of dichroic coating used to radially reflect light projected from the tubular element 212. In some embodiments, light within a predefined wavelength range may be radially reflected from the cone mirror 233 to produce a 360 degree ring of light while light within a second predefined wavelength range may be passed through the cone mirror 233 and projected out of the distal end of the probe 109 through the probe tip 215. In other embodiments, the cone mirror 233 may be configured with a silvered mask or other type of 100% radially reflective mask such that all light projected onto the cone mirror 233 will be radially reflected regardless of the wavelength.

As illustrated in FIG. 2B, the probe 109 includes a probe channel formed by the channels of the cone mirror 233, tubular element 212, optical guide 209 and/or lighting element 203. Disposed within at least a portion of the tubular element 212 is a lens system 227 configured to capture reflections of light the light radially reflected from the cone mirror 233 or passed through the cone mirror 233 when the light is projected onto a cavity surface. The reflections of light may be captured by the lens system 227 and guided through the inner channel of the probe 109 to an image sensor 230 disposed adjacent to the lighting element 203. The image sensor 230 may be coupled for data communications to a data processor. The data processor may be configured to construct a 3D image of the cavity surface, in dependence upon a sequence of images captured when the scanned cavity surface is illuminated by the scanning light and tracked positions of the probe 109 inferred from reflections of tracking illumination sensed by the tracking illumination sensors.

Referring next to FIG. 2C, is another example illustration of the probe 109 according to various embodiments. The probe 109, as shown in FIG. 2C, includes the lighting element 203, the light source 206, the optical guide 209, the tubular element 212, the illumination tube 218, the frustration mask 221, the filter element 224, a lens system 227, an image sensor 230, and/or other elements. As illustrated in FIG. 2C, the probe tip 215 may comprise a cone mirror 233, a second light source 239, and a distal window 236. The example of the probe as shown in FIG. 2C differs from the probe 109 as illustrated in FIG. 2B by including a second light source 239 at the distal end of the probe tip 215. The second lighting source 239 may be adjacently affixed adjacent to the cone mirror 233. The second light source 239 may comprise a laser, a light emitting diode (LED), or any other appropriate type of light source. The second light source 239 may generate light that is different from the light generated by the light source 206 adjacently disposed to the optical guide 209. For example, the light source 206 may comprise a blue LED and the second light source 239 may comprise a white LED. The blue LED may be used for scanning while the white LED may be used for video illumination.

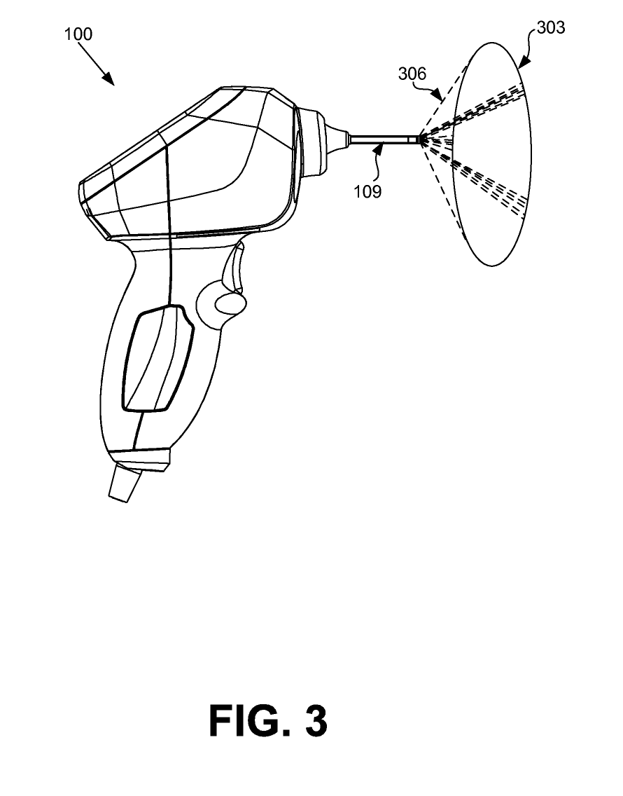

In some embodiments, the second light source 239 may be coupled to one or more wires (not shown) that are disposed along the probe and connected to a power source within the scanning device 100. In some embodiments, the wire(s) may be disposed within the probe channel extending from the first end of the probe 109 to the second end of the probe 109. Accordingly, the radially reflected light projected from the cone mirror 233 may still project a 360 degree ring 303 (FIG. 3) of light. However, the image sensor 230 may capture the images of a broken ring due to the location of the wire(s). In other embodiments, the wire(s) may be disposed along the outside of the probe 109. As such, the radially reflected light projected from the cone mirror 233 may be a broken ring due to the location of the wire. Accordingly, the image sensor 230 may capture the image of the broken ring due to the projected broken ring.

Moving on to FIG. 2D, shown is another example of the probe 109 according to various embodiments. The probe 109, as shown in FIG. 2D, includes a first lighting element 203, a first light source 206, the optical guide 209, the tubular element 212, the illumination tube 218, the frustration mask 221, the lens system 227, the image sensor 230, a second light source 239, and a second lighting element 241 and/or other elements. As illustrated in FIG. 2D, the probe tip 215 may comprise a cone mirror 233 and a distal window 236. The example of the probe 109 as shown in FIG. 2D differs from the probe 109 as illustrated in FIG. 2B by having a second light source 239 and a second lighting element 241 disposed around the tubular element 212 at the proximal end of the tubular element 212. As shown in FIG. 2D, the illumination tube 218 receives light generated by the second light source 239 disposed on the second lighting element 241. The second lighting element 241 and second light source 239 may be coupled to the probe 109 via a housing (not shown) which mounts the probe 109 to the body 103 of the scanning device 100. The second light source 239 may comprise a laser, a light emitting diode (LED), or any other appropriate type of light source. In some embodiments, the second lighting element 241 may comprise a printed circuit board (PCB) and/or other type of supporting element that may include the second light source 239. The second light source 239 may generate a light that is different from the light generated by the first light source 206. For example, the first light source 206 may be a blue LED which may be used for scanning a cavity surface while the second light source 239 may be a white LED which may be used for video illumination. The illumination tube 218 may be designed to aim the received illumination light generated by the second light source 239 at a desired angle for video illumination.

In some embodiments, the illumination tube 218 may comprise a single or double cladding. The cladding may be used to direct the angle of the light guided through the illumination tube 218. Accordingly, the cladding may be used to prevent light from escaping from the illumination tube 218 at undesired areas. Further, the cladding may approximately collimate the light as the light is guided through the tubular element 212.

In other embodiments, the probe 109 may be designed without an illumination tube 218. For example, the probe 109 may be configured such that the second light source 239 projects light into a fiber bundle (not shown) comprising multiple optical fibers for guiding light rather than an illumination tube 218. Accordingly, as with the illumination tube 218, light may be guided from a first end of the fiber bundle to a second end of the fiber bundle. In some embodiments, the fiber bundle may be tapered. The fiber bundle may be configured to aim the received illumination light generated by the second light source 239 at a desired angle for video illumination. In other embodiments, the light generated from the second light source 239 may be used without any type of guide. Accordingly, the light projected and generated by the second light source 239 is used as the video illumination without any type of guide (e.g., illumination tube 218, optical guide).

Turning now to FIG. 3, shown is an example of the scanning device 100 projecting an unbroken 360 degree ring 303 of light. The projected light 306 is the light that has been guided through the probe 109 via the optical guide 209 and tubular element 212 and radially reflected by the cone mirror 233. As illustrated in FIG. 3, the projected light 306 forms an unbroken 360 degree ring 303 of light. When the 360 degree ring 303 is projected into a cavity surface, such as, for example, an ear canal, the reflections may be captured by the lens system 227 (FIG. 2B) and guided to the image sensor 230 (FIG. 2B) within the scanning device 100 for processing and reconstructing.

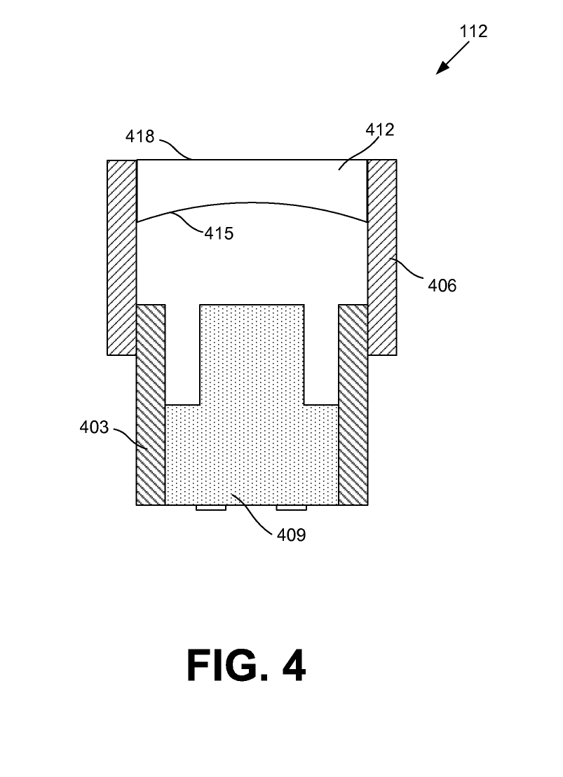

Referring next to FIG. 4, shown is an illustration of an example of the fan light element 112 according to various embodiments. The fan light element 112 may be included in, e.g., the scanning device 100 to produce a collimated line of light for imaging a surface. The fan light element 112 comprises a first structural tube 403, a second structural tube 406, a fan light source 409, and a fan lens 412. The first structural tube 403 and the second structural tube 406 may be elongated and may be made from metal, glass, plastic and/or any other type of material capable of structurally supporting the fan lens 412 and fan light source 409. In some embodiments, the first structural tube 403 may be affixed substantially adjacent to the second structural tube 406. In other embodiments, at least a portion of the first structural tube 403 may be affixed to the second structural tube 406 within an inner wall of the second structural tube 406. In other embodiments, at least a portion of the second structural tube 406 may be affixed to the first structural tube 403 within an inner wall of the first structural tube 403.

The fan lens 412 is disposed within the inner wall of the second structural tube 406. The fan lens 412 is a single lens element that combines the functions of a collimator and line generating lens. U.S. patent entitled “Laser Line Generation System” filed on Oct. 20, 1998 and assigned U.S. Pat. No. 6,069,748, provides a detailed description of the fan lens 412, and is incorporated by reference in its entirety. The fan lens 412 utilizes, in one direction, the natural divergence of light generated from the fan light source 409 to produce a fan line. In this direction, the fan lens 412 may have either negative, zero, or positive power in order to alter the angular spread of the light. In the other direction, positive optical power is introduced to collimate the diverging light exiting the fan light source 409 to produce a well-defined line of a predetermined thickness at any image distance.

The fan light source 409 may generate divergent light and is disposed within an inner wall of the first structural tube 403. The fan light source 409 may be a laser, a light emitting diode (LED), or any other appropriate type of light source. According to various embodiments, light generated by the fan light source 409 and projected onto the fan lens 412 may be emitted from the scanning device 100 in the form of a collimated fan line.

In some embodiments, the fan light element 112 may be assembled by affixing the fan lens 412 to the second structural tube 406. The fan lens 412 may be affixed to the second structural tube 406 with an adhesive material such as, for example, glue, epoxy, and/or other appropriate type of bonding agent. In other embodiments, the fan lens 412 may be affixed to the second structural tube 406 with at least one screw and/or other fastening device.

The fan lens 412 may be made of a glass or plastic material. The fan lens 412 comprises a first surface 415 including either an aspheric or a toroidal surface. The fan lens 412 further comprises a second surface 418 that is opposite the first surface 415. The second surface 418 may include a plano surface having no optical power or a cylindrical surface having additional optical power. The additional optical power may be either negative power or positive power. Positive optical power would reduce the divergence of the laser beam while negative optical power would increase the divergence of the divergent light.

Additionally, the fan light source 409 may be affixed to the first structural tube 403. The fan light source 409 may be affixed to the first structural tube 403 with an adhesive material such as, for example, glue, epoxy and/or other type of bonding agent. In other embodiments, the fan light source 409 may be affixed to the first structural tube 403 with at least one screw and/or other appropriate fastening device.

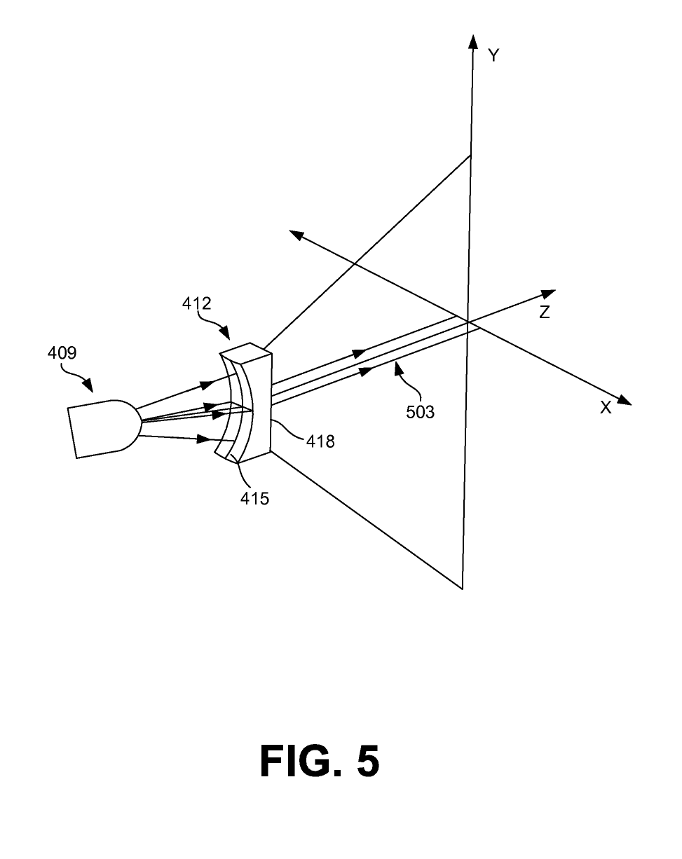

In some embodiments the first structural tube 403 and the second structural tube 406 may be positioned substantially adjacent to each other so that light generated by the fan light source 409 may be projected onto a first surface 415 of the fan lens 412 and emitted from a second surface 418 of the fan lens 412 that is opposite the first surface 415. In some embodiments, the light emitted from the second surface 418 of the fan lens 412 may be focused by increasing or decreasing the distance between the fan light source 409 and the fan lens 412. For example, if the first structural tube 403 is designed to fit within the second structural tube 406, the portion of the first structural tube 403 within the second structural tube 406 may be increased or decreased until the light emitted from the second surface 418 of the fan lens 412 is focused. In addition, the light may be further focused and aligned to thin the emitted collimated line by rotating the first structural tube 403 and/or the second structural tube 406 around the other to orient the fan lens 412 such that a positive optical power of the fan lens 412 corresponds to a slow axis of the diverging light and the negative optical power of the fan lens 412 corresponds to a fast axis of the diverging light as will be discussed in further detail with respect to FIG. 5.

When the first structural tube 403 is accurately positioned with the second structural tube 406 to form the desired focus and alignment, the first structural tube 403 may be affixed to the second structural tube 406 with a bonding material such as, for example, glue, epoxy, solder, welding agent and/or other appropriate bonding agent. In other embodiments, the first structural tube 403 may be affixed to the second structural tube 406 with at least one screw and/or other appropriate fastening device.

It should be noted the fan line element 112 described above comprising the single fan lens 412 and fan light source 409 within the first structural tube 403 and second structural tube 406 is one embodiment of the fan line element 112. In other embodiments, the fan line element 112 maybe comprise various types of optics such as Powell lens line generator, a diffractive optic line generator, a refractive optic line generator, a galvo mirror line generator, a spinning mirror line generator, and/or any other appropriate type of optics that may generate a fan line 503 when illuminated by a fan light source 409. In addition, in some embodiments, the fan light source 409 and optics such as, for example, the single fan lens 412, may be positioned within a single structural tube. In some embodiments, the fan light element 112 may comprise spacers which could be used to position the optics relative to the fan light source 409.

Further, although the fan light element 112 is configured above to projects a fan line 503 (FIG. 5), the fan light element 112 may be configured to projected other types of light such as structured or unstructured light. Accordingly, the scanning device 100 may be configured to scan an outer surface using the light projected from the fan light element 112.

Moving on to FIG. 5, shown is an illustration of an example of light being emitted from the fan lens 412 in a culminated line. The first surface 415 of the fan lens 412 is adjacent to the fan light source 409 and is configured to collimate the divergent light provided by the fan light source 409. The second surface 418 of the fan lens 412 spreads the light into a fan line 503. In this example, the Y-Z cross section of the first surface 415 of the fan lens 412 is concave. In addition, the Y-Z plane of the first surface 415 of the fan lens 412 has a substantially circular cross section with a radius of curvature. The second surface 418 of the fan lens 412 has an aspheric X-Z cross section. Optimally, the fan light source 409 is positioned at a distance such that the fan light source 409 is at the center of the radius of curvature of the first surface 415. The fan light source 409 may be configured to generate light that has different angular divergences in two orthogonal directions. The direction of the largest divergence angle of the light may be referred to as the fast axis and while the direction of the smaller divergence angle of light may be referred to as a slow axis. The fan lens 412 may be oriented such that a positive optical power corresponds to the slow axis and a negative optical power corresponds to the fast axis to produce the focused collimated fan line.

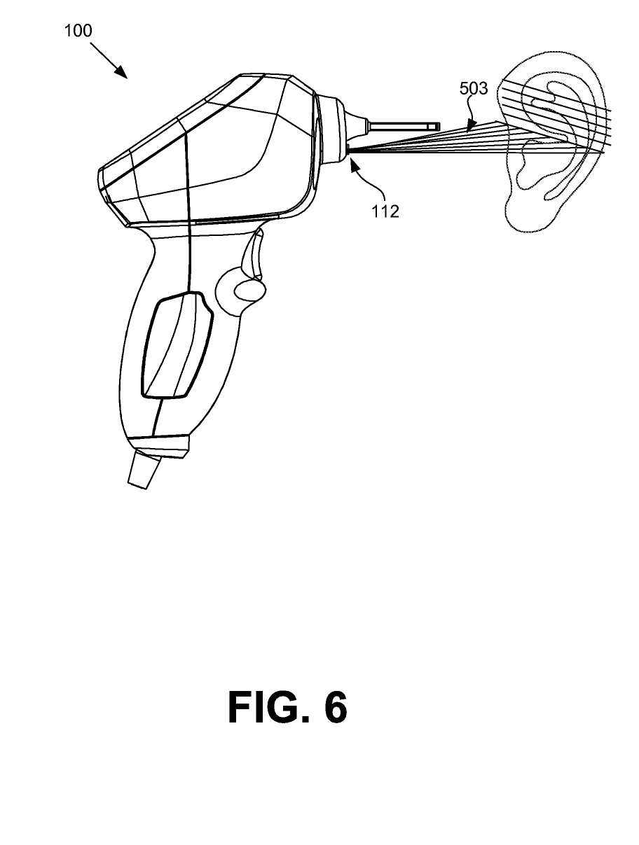

Turning now to FIG. 6, shown is an illustration of an example of the scanning device 100 emitting the fan line 503 (FIG. 5) for scanning a surface. In this example, the scanning device 100 is scanning the surface of an ear. However, it should be noted that the scanning device 100 may be configured to scan other types of surfaces. The fan light element 112 may be designed to emit a fan line 503 formed by projecting divergent light generated by the fan light source 409 onto the fan lens 412. As previously discussed, the fan lens 412 may comprise a single lens element that collimates and generated the fan line 503 that is emitted from the scanning device 100. As the fan line 503 is projected onto a surface, the lens system 227 (FIGS. 2B, 2C, and 2D) may capture reflections of the fan line 503. As previously discussed, the image sensor 230 (FIGS. 2B, 2C, and 2D) may use triangulation to construct an image of the scanned surface based at least in part on the reflections captured by the image sensor 230 via the lens system 227. Accordingly, the constructed image may be displayed on the display screen 118 (FIGS. 1A-1B) and/or other displays in data communication with the scanning device 100.

The fan light element 112 may be mounted within the scanning device 100. In some embodiments, an operator of the scanning device 100 may manually move the scanning device 100 along an axis relative to the surface to move the projection of the fan line 503 emitted from the fan light element 112 along the desired portion of the outer surface for imaging. As the fan line 503 moves as determined by the operator, the reflections of the fan line 503 projected onto the various portions of the surface are captured by the image sensor 230 via the lens system 227. Upon capturing the reflections of the fan line 503, the image sensor 230 may generate a 3D reconstruction of the surface based on the location of the captured reflections relative to a tracking fiducial. For example, the image sensor 230 may utilize a lookup table that defines the 3D position of the fan line 503 relative to the tracking fiducial.

In other embodiments, the scanning device 100 may be configured to include a refractive element, such as, for example, a mirror, that may be configured to move over the second surface 418 of the fan lens 412 when scanning the outer surface. Accordingly, by use of the refractive element, the scanning device 100 may be configured to scan a surface by adjusting the position of the refractive element over the fan lens 412 to adjust the angle of the fan line 503 projected from the fan light element 112. Accordingly, the operator would need to manually move the scanning device 100 to move the location of the fan line 503 for imaging. As the angle of the fan line 503 is adjusted by the refractive element, the reflections of the fan line 503 projected on the various portions of the surface are captured by the lens system 227 and used by the image sensor 230 for 3D reconstruction of the surface.

In other embodiments, the fan light element 112 may be affixed to a moveable mount within the scanning device 100 such that the operator does not have to move the scanning device 100 to scan the surface. The moveable mount may be configured to move the fan light element 112 along an axis relative to the surface so that the fan line 503 is projected onto the surface to be scanned as the moveable mount adjusts the position of the fan light element 112 relative to the scanning device 100. Accordingly, as the fan line 503 moves from the movement of the moveable mount, the reflections of the fan line 503 projected on the various portions of the surface are captured by the lens system 227 and used by the image sensor 230 for 3D reconstruction of the surface.

Moving on to FIGS. 7A and 7B, shown are illustrations of an example of the optical guide 209. The optical guide 209 comprises a plurality of optical fibers 703 for guiding light from the light source 206 to the tubular element 212 (FIGS. 2A-2B). Individual fibers are displaced substantially adjacent to other fibers such that the plurality of optical fibers 703 surround a bundle channel 712 extending from a first end of the optical guide 209 to a second end of the optical guide 209. Although the optical guide 209 displayed in FIG. 7A illustrates only a section of optical fibers 703 it should be understood that optical fibers 703 surround the entire optical guide 209.

In one embodiment, the optical guide 209 may be created by loosely inserting the optical fibers 703 between two glass, acrylic, and/or plastic cylinders where a first cylinder is inserted inside the a second cylinder and each extend from the first end to the second end of the optical guide 209. Accordingly, the optical guide 209 comprises an inner wall formed by the first cylinder and an outer wall formed by the second cylinder. The inner wall of the first cylinder defines the bundle channel 712. After the optical fibers 703 are inserted between the two cylinders, the optical guide 209 may be heated to fuse at least a portion of the optical fibers and pulled to taper the optical fibers 703 and, consequently, the optical guide 209. The optical fibers 703 are fused together to minimize and substantially eliminate any gaps between the optical fibers 703 at the portion in which they are fused. It should be noted that fewer gaps between the optical fibers 703 minimize the risk of light escaping from the individual optic fibers 703.

The optical guide 209 may be tapered such that the diameter of the first end as shown in FIG. 7A is greater than the diameter of the second end as shown in FIG. 7B. Accordingly, the optical fibers 703 are also tapered such that a first end terminal of individual fibers is greater than a second end terminal of the individual fibers. In addition, while the optical guide 209 shown in FIGS. 7A and 7B is frustroconical in shape, it should be noted that the optical guide 209 may be shaped as a cone, a bell, and/or other type of tapered form. For example, the optical guide 209 may taper in a linear fashion from the first end to the second end as illustrated in FIGS. 7A and 7B. In other implementations, the optical guide 209 may taper in a non-linear fashion, such as, e.g., a bell shape such as that shown in FIGS. 2A-2D.

In another embodiment, the optical guide 209 may be created using a predefined mold. Multiple optical fibers 703 may be inserted into a predefined mold to be glued and/or heated together to form a molded tapered bundle of the optical fibers. The mold of the tapered bundle creates a bundle channel 712 extending from the first end of the optical guide 209 to the second end of the optical guide 209. At least a portion of the optical fibers may surround and/or potentially define the channel. The channel may also be tapered along with the optical fibers 703. The mold may be defined with a tapering such that the diameter of the first end as shown in FIG. 7A is greater than the diameter of the second end as shown in FIG. 7B. Accordingly, the optical fibers are also tapered such that a first end terminal of individual fibers is greater than a second end terminal of the individual fibers.

As previously discussed the first end of the optical guide 209 may be disposed adjacent to the light source 206 (FIGS. 2A-2B) while the second end of the optical guide 209 may be disposed adjacent to the proximal end of the tubular element 212 (FIGS. 2A-2B). The light generated by the light source 206 may travel through the optical fibers 703 to the tubular element 212 and/or the illumination tube 218 (FIG. 2B) according to various embodiments. The use of a tapered bundle of optical fibers 703 minimizes the amount of escaping light as the light is guided through the optical guide 209.

The bundle channel 712 of the optical guide 209 may be used to guide reflections captured by the lens system 227 (FIG. 2B) disposed within at least a portion of the tubular element 212 and possibly the channel of the optical guide 209. The reflections are from light guided through the optical guide 209 from the light source 206 and subsequently projected onto a cavity surface. The reflections are guided through the axial length of the bundle channel 712 defined by the optical guide 209 and received by an image sensor 230 adjacent to the first end of the optical guide 209. The image sensor 230 captures and reconstructs images of the scanned and/or illuminated surface.

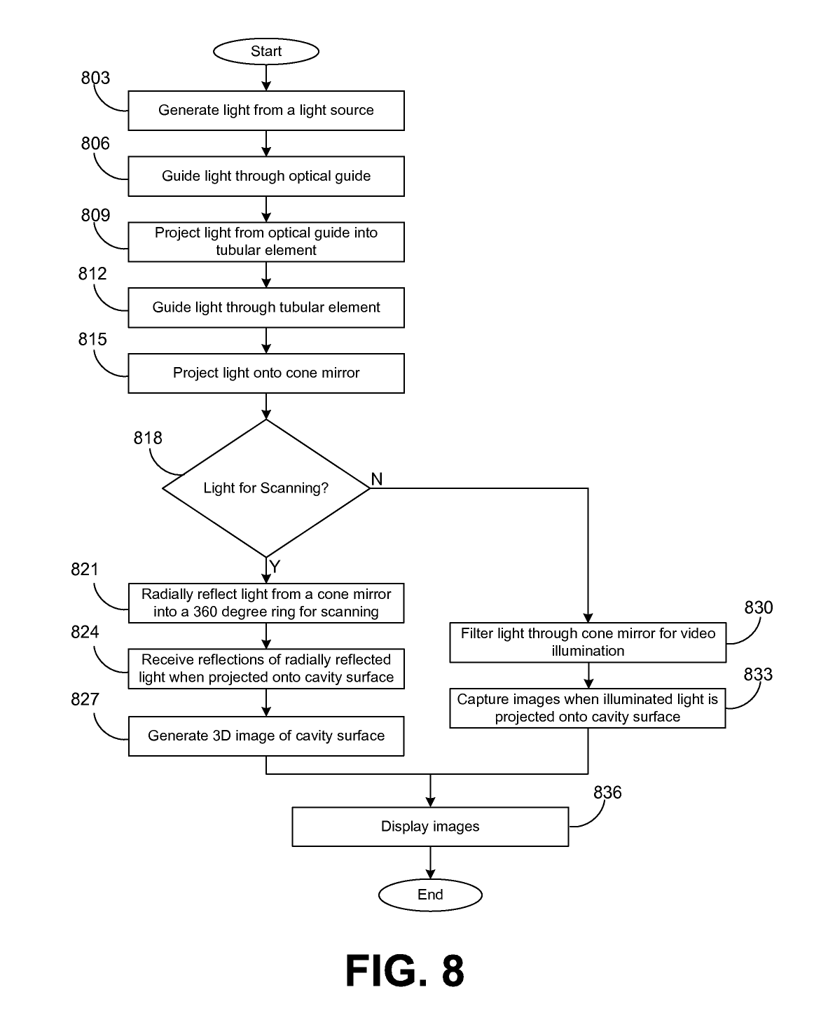

Referring next to FIG. 8, shown is a flowchart 800 illustrating an example of a method for scanning and displaying the scanned images of a cavity surface. Beginning with 803, light is generated by a light source 206 (FIGS. 2A-2D) disposed on the proximal end of the probe 109. The light source 206 may be a LED, a laser, or any other type of light generating source. The light generated by the light source 206 is projected into the first end of the optical guide 209 (FIGS. 2A-2D).

At 806, the light is guided through the optical guide 209 from the first end of the optical guide 209 to the second end of the optical guide 209. As previously discussed, the optical guide 209 is comprised of multiple optical fibers 703 (FIGS. 7A-7B) comprising a bundle of optical fibers surrounding a bundle channel 712 (FIGS. 7A-7B). Light received from the light source 206 at the first end of the optical guide 209 may be guided through individual fibers to the second end of the optical guide 209. Accordingly, because the light is guided through individual fibers rather than an open structure, the amount of escaping light is minimized. Additionally, the optical guide 209 is tapered further minimizing coupling loss when the optical guide 209 is adjacently disposed at the proximal end of the tubular element 212 (FIGS. 2A-2D).

At 809, light guided through the optical guide 209 is projected into the proximal end of the tubular element 212. Further, the light is received between the inner wall and outer wall of the tubular element 212. At 812, light received at the proximal end of the tubular element 212 is guided through the tubular element 212. As previously discussed, the tubular element 212 may include cladding on at least the inner or outer wall of the tubular element 212. The cladding configuration minimizes the amount of light escaping while being guided through the tubular element 212. Additionally, the cladding facilitates the proximal collimation of the light guided through the tubular element 212. In addition, the tubular element 212 may comprises a frustration mask 221 (FIGS. 2B-2D) which may absorb light escaping from the tubular element 212.

At 815, light guided through the tubular element 212 is projected onto the cone mirror 233 (FIGS. 2B-2D) adjacently disposed on the second end of the tubular element 212. As determined at 818, if the light is scanning light based on its wavelength proceed to 821. Otherwise, proceed to 830. As previously discussed, light with varying wavelengths may be alternately guided through the scanning probe 109. One light may be used for scanning while the other may be used for video illumination.

At 821, the light projected onto the cone mirror 233 from the second end of the tubular element 212 may be radially reflected from the cone mirror 233 into a 360 degree ring 303 (FIG. 3) of light. The cone mirror 233 may be coated with a dichroic coating or other type of coating which may reflect light within a certain predefined wavelength. For example, a silvered coating may reflect 100% of light projected while a dichroic coating may only reflect light with wavelengths, for example, of about 450 nm or less. At 824, when the 360 degree ring 303 of light is projected onto a cavity surface, reflections may be captured by the image sensor 230 (FIGS. 2B-2D) at the proximal end of the probe 109 via the lens system 227 disposed within at least a portion of the tubular element 212. At 827, the captured reflections may be processed to construct a shape and 3D image of the scanned cavity surface.

At 830, the light received at the cone mirror 233 which is within a different predefined wavelength that is not used for scanning is filtered through the cone mirror 233 for video illumination. At 833, video images are captured by the sensor when the illuminated light is projected onto the cavity surface.

At 836, the captured and generated video and 3D images are displayed. The image sensor is in data communication with the display screen 118 mounted upon the body 103 of the scanning device 100. The display screen 118 may separately or simultaneously display the real-time constructions of 3D images of the scanned cavity and the video images. Additionally, any other displays in data communication with the image sensor 230 may also display the constructed images separately or simultaneously.

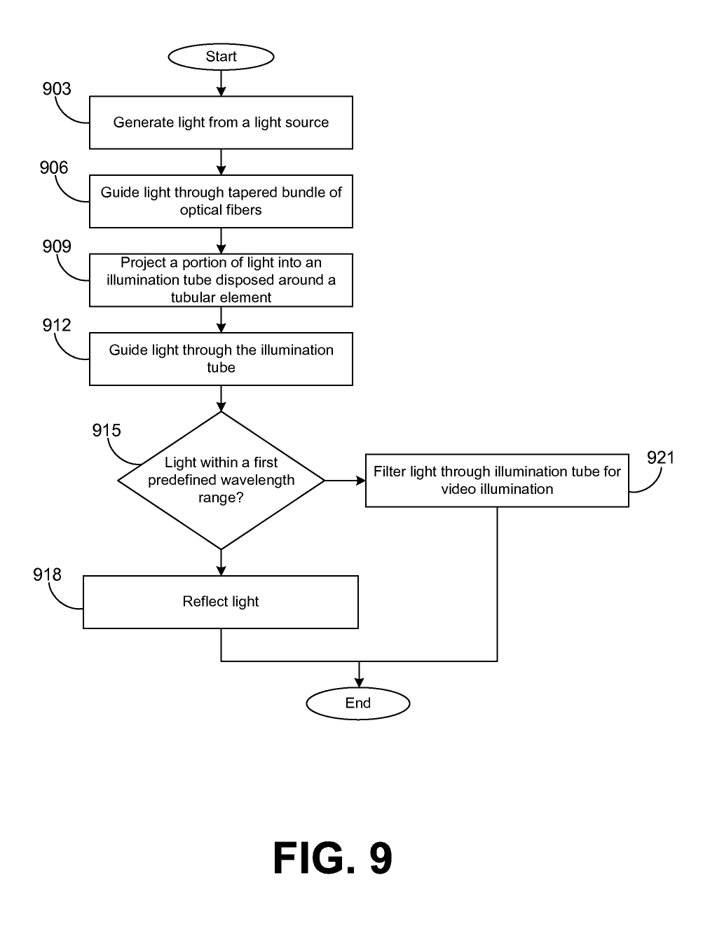

Referring next to FIG. 9, shown is a flowchart 900 illustrating an example of a method for projecting light for video illumination through the illumination tube 218. Beginning with 903, light is generated by a light source 206 (FIGS. 2A-2D) disposed on the proximal end of the probe 109 (FIGS. 1A-1B). The light source 206 may be a LED, a laser, or any other type of light generating source. The light generated by the light source 206 is projected into first end of the optical guide 209 (FIGS. 2A-2D).

At 906, the light is guided through the optical guide 209 from the first end of the optical guide 209 to the second end of the optical guide 209. As previously discussed, the optical guide 209 is comprised of multiple optical fibers 703 (FIGS. 7A-7B) comprising a bundle of optical fibers surrounding a bundle channel 712 (FIGS. 7A-7B). Light received from the light source 206 at the first end of the optical guide 209 may be guided through individual fibers to the second end of the optical guide 209. Accordingly, because the light is guided through individual fibers rather than an open structure, the amount of escaping light is minimized. Additionally, the optical guide 209 is tapered further minimizing coupling loss when the optical guide 209 is coupled to the tubular element 212.

At 909, light guided through the optical guide 209 may be received at a proximal end of the illumination tube 218 (FIGS. 2B & 2D) that is disposed around the tubular element 212. At 912, the light is guided through the illumination tube 218. As previously discussed, the illumination tube 218 may comprise a filter element 224 (FIG. 2B-2C) for passing light through when the light is within a desired wavelength range. Accordingly, at 915, if the light is within a first predefined wavelength, the light will be reflected as stated at 918. Otherwise, as stated at 921, the light will pass through the filter element 224 and the light will be projected from the illumination tube 218.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A scanning probe comprising:
a tubular element comprising an inner wall, an outer wall, and a frustration mask, the inner wall and the outer wall extending from a first end of the tubular element to a second end of the tubular element, the frustration mask being disposed on the outer wall of the tubular element and extending at least a partial length from the first end of the tubular element to the second end of the tubular element, the tubular element being designed to:
guide light from the first end of the tubular element to the second end of the tubular element and between the inner wall and the outer wall, the light being received from a light source; and
approximately collimate the light based at least in part on the frustration mask.

2. The scanning probe of claim 1, further comprising a cone mirror fixed at the second end of the tubular element, the cone mirror designed to radially reflect the light guided to the second end of the tubular element when the light is within a predefined wavelength range.

3. The scanning probe of claim 2, wherein the light that is radially reflected from the cone mirror forms a 360 degree ring.

4. The scanning probe of claim 2, wherein at least a portion of the cone mirror comprises a dichroic coating.

5. The scanning probe of claim 1, wherein the light source is a light emitting diode (LED).

6. The scanning probe of claim 1, further comprising an optical guide coupled to the light source, wherein the optical guide guides light generated by the light source to the first end of the tubular element.

7. The scanning probe of claim 6, further comprising an illumination tube disposed around the first end of the tubular element, wherein the optical guide guides at least a portion of the light generated by the light source to a proximal end of the illumination tube.

8. The scanning probe of claim 7, wherein a distal end of the illumination tube comprises a filter element configured to reflect the at least a portion of the light that is within a predefined wavelength range and pass the at least a portion of the light that is within a different predefined wavelength range.

9. The scanning probe of claim 8, wherein the predefined wavelength range is about 450 nm and less.

10. The scanning probe of claim 8, wherein the different predefined wavelength range is about 500 nm and above.

11. The scanning probe claim 7, wherein the illumination tube is disposed around the proximal end of the tubular element over at least a portion the frustration mask of the tubular element.

12. The scanning probe of claim 1, further comprising a lens system disposed within at least a portion of the inner wall of the tubular element, wherein the lens system is configured to capture a reflection from the light that has been radially reflected when the light that has been radially reflected is projected onto a cavity surface.

13. A scanning probe comprising:
a tubular element comprising an inner wall and an outer wall, the inner wall and the outer wall extending from a first end of the tubular element to a second end of the tubular element, at least a portion of the outer wall comprising a first cladding, at least a portion of the inner wall comprising a second cladding, and the tubular element being designed to:
guide light received at the first end of the tubular element to the second end of the tubular element, the light being guided between the inner wall and the outer wall; and
approximately collimate the light guided to the second end of the tubular element.

14. The scanning probe of claim 13, further comprising a cone mirror fixed at a second end of the tubular element, the cone mirror being designed to radially reflect the light projected from the second end of the tubular element onto the cone mirror when the light is within a first predefined wavelength range.

15. The scanning probe of claim 13, wherein the light is generated by a light source.

16. The scanning probe of claim 15, wherein the light source is a light emitting diode (LED).

17. The scanning probe of claim 15, further comprising an illumination tube disposed around the first end of the tubular element, wherein a proximal end of the illumination tube receives at least a portion of the light generated by the light source.

18. The scanning probe of claim 17, further comprising a filter element disposed on the illumination tube, the filter element being designed to:
reflect the at least a portion of the light received from at the proximal end of the illumination tube when the at least a portion of the light is within a first predefined wavelength range; and
pass the at least a portion of the light when the at least a portion of the light is within a second predefined wavelength range.

19. The scanning probe of claim 17, further comprising a frustration mask disposed between the tubular element and the illumination tube disposed around the tubular element.

20. The scanning probe of claim 13, further comprising an optical guide coupled to a light source, wherein the optical guide transmits light generated by the light source to the first end of the tubular element.

21. The scanning probe of claim 13, further comprising a frustration mask disposed around at least a portion of the tubular element.

22. The scanning probe of claim 13, wherein the light is generated from a first light source, and further comprising an illumination tube disposed around the first end of the tubular element, wherein a proximal end of the illumination tube receives illuminating light generated by a second light source.

23. The scanning probe of claim 22, wherein the illumination tube comprises a cladding.

24. The scanning probe of claim 22, wherein illumination tube is designed to guide the illuminating light generated by the second light source to a distal end of the illumination tube.

25. A method for scanning a cavity surface, the method comprising:
transmitting light from a first end of a tubular element to a second end of the tubular element, the light being approximately collimated in the tubular element based at least in part on a frustration mask extending along at least a portion of an outer wall of the tubular element;
radially projecting the light from the second end of the tubular element onto a cavity surface; and
capturing a reflection from the radially projected light via a lens system.

26. The method of claim 25, wherein radially projecting the light further comprises:
projecting the light from the second end onto a cone mirror; and
radially reflecting the light projected onto the cone mirror when the light is within a predefined wavelength range.

27. The method of claim 26, further comprising filtering the light through the cone mirror when the light is within a different predefined wavelength range.

28. The method of claim 25, wherein the tubular element comprises a double cladding, and wherein the light being approximately collimated in the tubular element is further based at least in part on the double cladding.

29. The method of claim 28, wherein transmitting the light through the tubular element further comprises guiding the light through a core created within the double cladding of the tubular element.

30. The method of claim 25, further comprising generating the light from a light source.

31. The method of claim 30, wherein a portion of the light generated by the light source is received by an illumination tube disposed around the first end of the tubular element.

32. The method of claim 31, further comprising:
reflecting the portion of the light received by the illumination tube when the light is within a predefined wavelength range; and
filtering the portion of the light through the illumination tube when the light is within a different predefined wavelength range.

33. The method of claim 30, wherein the light source comprises a plurality of light sources, and further comprising alternately generating the light from a first light source and a second light source, wherein the light generated by the first light source is within a predefined wavelength range and the light generated by the second light source is within a different wavelength range.

34. The method of claim 33, wherein the first light source and the second light source are LEDs.
